(19) United States
(12) Patent Application Publication
(10) Pub. No.: US 2014/0335185 A1
(43) Pub. Date: Nov. 13, 2014

(54) NOVEL MICROCARRIER BEADS

(71) Applicant: National University of Singapore, Singapore (SG)

(72) Inventors: Eng San Thian, Singapore (SG); Yong Yao Jason Feng, Singapore (SG); Kok Yen Jerry Chan, Singapore (SG); Ying Hsi Jerry Fuh, Singapore (SG)

(73) Assignee: National University of Singapore, Singapore (SG)

(21) Appl. No.: 14/268,122

(22) Filed: May 2, 2014

(30) Foreign Application Priority Data
May 10, 2013 (GB) 1308389.4

(51) Int. Cl. C12N 5/077; A61K 35/32; A61K 33/42; C12N 5/00

(52) U.S. Cl. CPC C12N 5/0654; C12N 5/0075; A61K 35/32; A61K 33/42; C12N 2506/1392; C12N 2533/18; C12N 2531/00
USPC 424/489; 435/402; 435/366; 424/93.7; 424/602; 435/377; 264/13; 428/402

(57) ABSTRACT

The invention relates to a novel microcarrier bead; a method for producing same; a therapeutic comprising said microcarrier bead and attached thereto or grown thereon at least one selected cell or tissue type; a method for making said therapeutic; and a method of treatment involving the use of said microcarrier bead or said therapeutic.

### CROSS-REFERENCE TO RELATED APPLICATIONS

The present application claims the priority of U.K. Provisional Patent Application Serial No. 1308389.4, which was filed on May 10, 2013, and is hereby incorporated by reference.

### FIELD OF THE INVENTION

The invention relates to a novel microcarrier bead; a method for producing same; a therapeutic comprising said microcarrier bead and attached thereto or grown thereon at least one selected cell or tissue type; a method for making said therapeutic; and a method of treatment involving the use of said microcarrier bead or said therapeutic.

### BACKGROUND OF THE INVENTION

Tissue engineering involves the use of a number of technologies relating to cells, engineering, materials and biochemical and physio-chemical factors to improve or replace biological functions. It also involves understanding the principles of tissue growth, and applying this knowledge to produce functional replacement tissue for clinical use. Tissue engineering uses living cells examples of which include living fibroblasts in skin replacement or repair and living chondrocytes in cartilage repair. There are many potential forms of cell therapy, including: the transplantation of stem cells or progenitor cells that are autologous (from the patient) or allogeneic (from another donor); the transplantation of mature, functional cells (Cell Replacement Therapy); and the application of modified human cells that are used to produce a needed substance (cell-based gene therapy).

A specific example of an area of research and of clinical interest is that of bone-related diseases and injuries. It requires a successful tissue-engineered solution that can replace and repair damaged bone or cartilage tissue both anatomically and functionally.

Although several achievements have been made in bone regenerative medicine, there is still much improvement to be made. Indeed, despite advances in the field, no adequate bone substitute has been developed and hence large bone defects/injuries still represent a major challenge for orthopaedic and reconstructive surgeons.

One reason for the delayed progress is that many tissue engineering protocols involve the use of cells or tissue that are implanted or seeded into artificial structures, or scaffolds, in order to promote three-dimensional tissue formation. Scaffolds are often critical, both ex vivo as well as in vivo, to represent the in vivo milieu (such as extracellular attachment, promote intracellular signaling, or the exertion of mechanical or biological influence to modify cell behavior) and to allow cells to influence their own microenvironments.

Scaffolds have to meet specific functional and technical requirements, depending upon their application, for example: porosity, to facilitate seeding and promote diffusion of nutrients and intra-cellular signals; biodegradability; strength, especially where the engineered tissue is to be subject to mechanical load forces. However, existing scaffolding technologies, even with cell augmentation techniques, have yet to yield sustained and reliable long-term results. Furthermore, the sheer number of cells required for a stem-cell augmented tissue regeneration strategy necessitates the development of an efficient method that is able to expand viable stem cells, and subsequently direct them to their intended function.

Additionally, scaffolds have to be customised to fit into the defect site that, more often than not, is irregular and complex, which is time consuming and costly. Furthermore, implantation of such scaffolds normally requires invasive surgical techniques that expose the patient to possible risks of infection. Injectable systems are thus desirable for such applications. Current systems include a direct injection of stem cells or an injectable gel-like bioactive material or a combination of both. However, such systems are prone to failure due to stem cell migration away from the defective site or insufficient time for the bioactive material to integrate with the host tissue for any beneficial effect to take place.

It is therefore necessary to incorporate a solid phase within this gel, one that will form the bulk of the scaffold, and remain in the defective site for a considerable period of time after the gel has been resorbed.

A bioreactor in tissue engineering is a device that attempts to simulate a physiological environment in order to promote cell or tissue growth in vitro typically for subsequent use in vivo. Mammalian cell growth in bioreactors can be facilitated with microcarrier beads, allowing for increased yields. Microcarrier beads are able to provide a 3D microenvironment with high surface area to volume ratio for cell adhesion and proliferation. Bioreactors and microcarrier beads have been described in the prior art (U.S. Pat. Nos. 5,073,491, 5,175,093, WO9314192).

However, existing microcarrier beads are most often constructed of porous gelatin, which disadvantageously exhibits random pore orientation and unpredictable pore interconnectivity. Further, existing microcarrier beads do not provide protection from an agitated fluid environment which is needed to achieve high cell yields. In the context of tissue engineering applications, existing microcarrier beads are often large in diameter (typically 2-3 mm), which leads to difficulties upon implantation and tissue integration, often resulting in poor cell proliferation and reduced cell viability inevitably leading to poor tissue differentiation and repair.

Herein disclosed is a novel micrometre-sized, phase-pure microbead with a regular porous structure. Advantageously, the microcarrier beads disclosed herein have excellent osteo-conductivity and chemical similarity to the mineral phase of natural bone, making them an ideal choice for bone regeneration and remodeling. Further, said microcarrier beads have high thermal stability permitting them to be easily sterilized. However, those skilled in the art will appreciate that the microcarrier beads of the invention are not limited to use in relation to bone or cartilage but also have application in dentistry, particularly in the treatment of periodontal defects.

As an example of proof of concept there is herein demonstrated a method to obtain Mesenchymal Stem Cells (MSCs) with enhanced osteogenic potency using the microcarrier beads according to the invention, which could be used in direct bone implant science.

### STATEMENTS OF INVENTION

According to a first aspect of the invention there is therefore provided a microcarrier bead made from apatite and characterised by one or more, including any combination, of the following features:
a) micrometre-sized;
b) a regular porous structure;
c) rough surface;
d) substantially spherical;
e) osteo-conductivity;
f) chemical similarity to the mineral phase of natural bone; and
g) have high thermal stability permitting them to be easily sterilized.

In a preferred embodiment of the invention there is provided a plurality of said microcarrier beads.

Reference herein to phase-pure refers to the purity of the microbeads, in phase-pure microbeads, the apatite is entirely made up of one sort of apatite such as, for example, pure hydroxyapatite (HA) with the chemical formula [Ca₁₀(PO₄)₆(OH)₂]; there are no inclusions of other phases such as tricalcium phosphate (TCP), tetracalcium phosphate (TTCP), biphasic calcium phosphate (BCP), etc.

In the invention said apatite may be phase-pure or include a number of different apatites and so not be phase-pure.

Apatite is a well-known group of phosphate minerals, comprising but not limited to hydroxylapatite, fluorapatite and chlorapatite, which are named for their high concentrations of OH⁻, F⁻, Cl⁻ or ions, respectively, in the crystal.

In a preferred embodiment of the invention, any form of apatite can be used, such as, but not limited to pure hydroxyapatite. However, silicon-substituted apatite, silver-substituted apatite, magnesium-substituted apatite may also be used to work the invention. Ideally, the choice of apatite is dependent upon the particular application. A preferred choice of apatite is a stoichiometric apatite which is a synthetic apatite with a Ca/P atomic ratio that approaches 1.67.

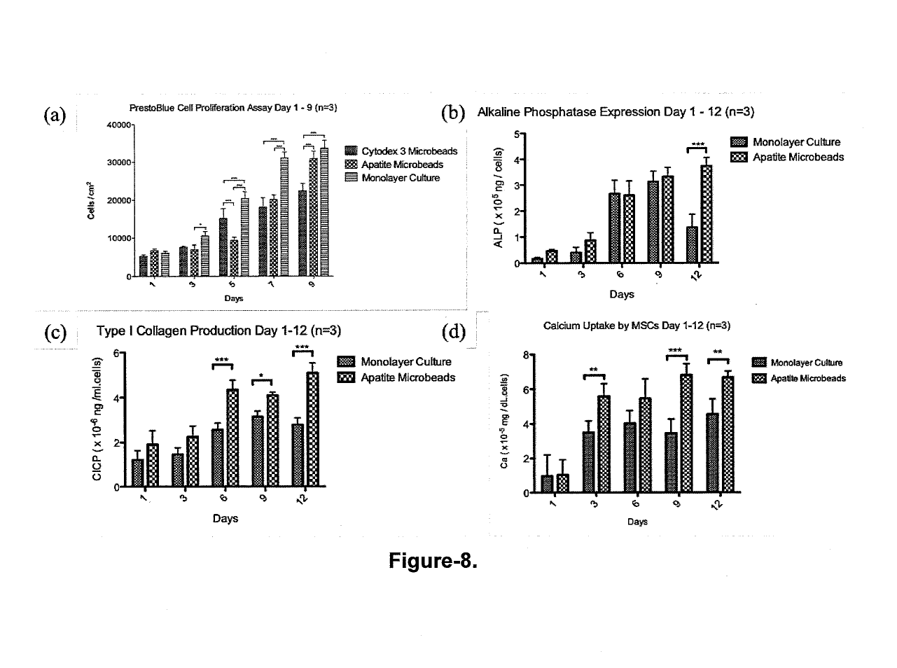

Reference herein to Osteo-conductivity is reference to a material that is able to support bone formation over its surface. In the case of the current in-vitro study, we have demonstrated this property by showing that ECM is laid over the material such that an aggregation of microbeads is formed by day 9 (FIG. 7c). In addition, collagen I (the organic phase of bone) production increased over the days in culture (FIG. 8c) whilst calcium uptake shows that MSCs proliferating on the microbeads are converting soluble calcium ions from the surrounding media and depositing them onto the microbeads (FIG. 8d).

In yet a preferred embodiment of the invention said beads are between 100-800 μm diameter including all 1 μm intervals there between, ideally 200-600 μm, more ideally still 400-500 μm such as 450 μm.

In yet a preferred embodiment of the invention said beads have a regular pore size as observed by SEM.

In yet a preferred embodiment of the invention said beads can withstand temperatures up to 1500° C. for up to 10 hours and in particular 1150° C. for 2 hours.

Ideally, said microcarrier beads also have osteogenic potency.

Reference herein to Osteogenic potency is reference to the ability of the microbeads to support osteogenic differentiation. MSCs seeded onto the microbeads are induced to differentiate down the osteogenic lineage by changing culture media from D10 to bone induction media (BM). Osteogenic differentiation is assessed by ALP expression (FIG. 8b). The current results show that ALP levels increases throughout the days, demonstrating that MSCs are actively differentiating. The type of differentiation is determined phenotypically by assessing collagen I production and calcium uptake.

Accordingly, in one aspect the invention concerns the provision of 3-dimensional porous apatite microcarrier beads that mimic the chemical and morphological structure of physiological bone.

The development of a novel porous microbead can be used as part of a strategy to assist and improve existing methods of orthopaedic surgery. The ease of implantation makes these microbeads a less costly and time consuming solution compared to the current scaffold solutions.

According to a further aspect of the invention there is provided a method for making microcarrier beads comprising:

a) adding apatite to a solution of alginate and allowing same to disperse to form a suspension;
b) extruding said suspension drop-wise through a droplet device;
c) exposing said extruded droplets to calcium chloride (CaCl₂) solution;
d) washing said beads to remove said CaCl₂ solution and dispersing same;
e) hardening the beads in a solution of alcohol;
f) drying the beads; and
g) sintering the beads to burn of the alginate.

In a preferred method if the invention any form of apatite can be used, such as, but not limited to pure hydroxyapatite, Silicon-substituted apatite, Silver-substituted apatite, Magnesium-substituted apatite, ideally, the choice of apatite is dependent upon the particular application. A preferred choice of apatite is a stoichiometric apatite which is a synthetic apatite with a Ca/P atomic ratio that approaches 1.67. More ideally still, a single type of apatite is used to generate a phase-pure microcarrier bead. Alternatively, more than one type of apatite may be used to generate a microcarrier bead that is not phase-pure.

In yet a further preferred method of the invention a salt of alginic acid was used as the alginate, having a M/G ratio of 1.56 (61% mannuronic, 39% guluronic acid). Preferably this alginate is dissolved in deionised water until total homogenisation at a concentration of 0.03 g/ml.

Alternatively, other types of alginate may be used such as, for example, and without limitation Sodium alginate (W201502 Aldrich), Alginic acid sodium salt (180947 Aldrich) or Alginic acid calcium salt from brown algae (21054 Aldrich).

Ideally, apatite powder was added to the alginate solution in a proportion of 50 wt % until thorough dispersion. Preferably, Camphene, serving as a porogen, is added in a proportion of 10-30 wt % to increase the desired porosity level of the microbeads.

Alternatively, other porogens can be used such as, for example, and without limitation Camphene ideally at 10-50 wt. %, or Starch ideally at 10-50 wt. % or Gelatine ideally at 10-50 wt. %.

More preferably, under part b) above the suspension was extruded drop-wise through an air-pressure assisted, electrical valve controlled droplet device (drop-on-demand).

More preferably, under part c) above said droplets were allowed to fall onto a 0.5 M calcium chloride solution.

More preferably, still, under part d) above the microbeads were washed twice in de-ionised water before immersing in 20 vol. % Tween 20 to disperse the microbeads.

Yet more preferably, under part e) the beads were immersed in isopropyl alcohol.

Isopropyl alcohol is ideally obtained commercially. Listed here are the common types of isopropyl alcohol that can be used: Isopropyl alcohol (W292907 Aldrich), Isopropyl alcohol (I9030 Sigma-Aldrich), Isopropyl alcohol (I9782 Sigma-Aldrich), 2-Propanol (I9516 Sigma), 2-Propanol (278475 Sigma-Aldrich).

Yet more preferably, under part f) the beads were subjected to a multi-stage sintering process which took the temperature to 1150° C. for 2 h in air to burn-off the alginate, thereby producing phase-pure sintered apatite microbeads.

According to a further aspect of the invention there is provided a therapeutic comprising a microcarrier bead according to the invention bead and attached thereto or grown thereon at least one selected cell or tissue type.

In a preferred embodiment of this aspect of the invention said cell or tissue is a stem cell or a progenitor cell with potential to differentiate into a selected tissue type when exposed to an induction or differentiation medium. Preferably said stem cell is a human cell, ideally an embryonic, fetal or adult stem cell, more ideally still said cell is an induced pluripotent stem cell.

Most preferably the therapeutic of the invention is particularly useful for the repair of craniomaxillofacial defects, wrist fractures, spinal fusion procedures and periodontal defects.

According to a further aspect of the invention there is provided a method for making a therapeutic comprising;
a) mixing microcarrier beads in accordance with the invention with at least one selected cell or tissue type in solution to form a suspension;
b) agitating said suspension to encourage said cells or tissue to attach to said beads;
c) culturing said cell attached beads to encourage growth of said cells or tissue; and
d) adding said cell-attached beads to a carrier gel.

In a preferred embodiment of the method of the invention said beads are sterilized prior to use, ideally using autoclave typically, but not exclusively, used at 124° C. for 30 min.

In yet a further preferred embodiment of the invention said solution comprises D10 medium (DMEM supplemented with 10% foetal bone serum and 1% penicillin streptomycin). Moreover said selected cell or tissue type is a stem cell or a progenitor cell with potential to differentiate into a selected tissue type when exposed to an induction or differentiation medium. Preferably said stem cell is a human cell, ideally an embryonic, fetal or adult stem cell, more ideally still said cell is an induced pluripotent stem cell. Ideally said stem cells were added at a density of 1.0×10⁵ cells/ml to 2 mg/ml of apatite microbeads.

More preferably still said agitation involves gentle intermittent agitation such as, for example, subjected to cycle at 10 rpm for 5 min followed by 30 min rest, for 12 h, although other cycles known to those skilled in the art may be used.

Yet more preferably still, step c) above involves exposing said cells or tissue to induction or differentiation medium such as bone induction medium (D10 medium supplemented with 10 mM β-glycerophosphate, 10⁻⁸M dexamethasone and 0.2 mM ascorbic acid).

According to a further aspect of the invention there is provided a method of treatment involving the use or administration of the microcarrier beads and/or the therapeutic of the invention.

Most preferably the invention is particularly useful for the treatment or repair of cranio-maxillofacial defects, wrist fractures, spinal fusion procedures and periodontal defects.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The Invention will now be described in greater detail with reference to the Examples below and to the drawings in which:

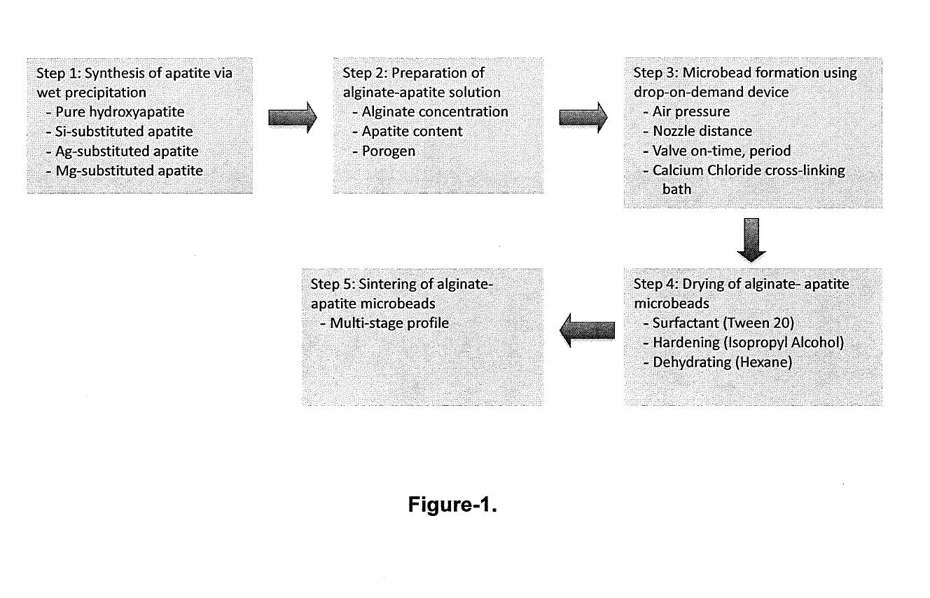

FIG. 1. Schematic summary describing the process of fabricating phase-pure apatite microbeads.

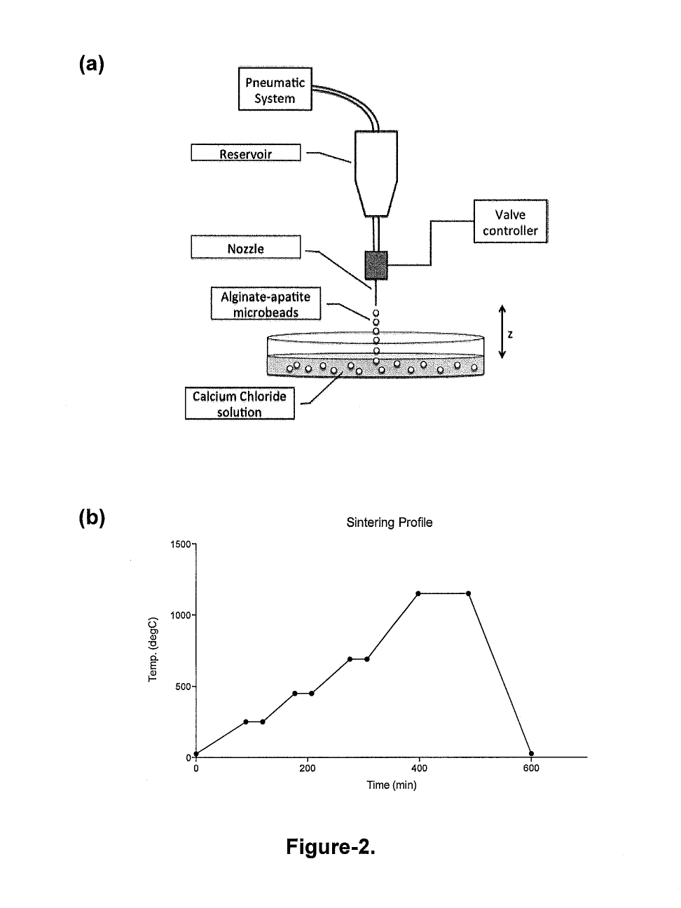

FIG. 2. (a) Schematic diagram of the drop-on-demand device, whereby the alginate-apatite solution is loaded into the reservoir, and extruded through a 250 μm sized nozzle. Valve control is set at 5 ms open, 95 ms closed. Pressure is varied through a range of 2.5-4.5 bar to achieve single droplet formation. These processing parameters produce 300-400 μm sized spherical microbeads at a rate of up to 600 microbeads per minute. (b) Sintering profile for obtaining phase-pure sintered apatite microbeads. From room temperature, the microbeads are heated to a temperature of 250° C. at a rate of 2.5° C./min. This temperature is held for 30 min, then increased to 450° C. at a rate of 3.5° C./min, and held for another 30 min. A third hold temperature of 690° C. is achieved at 3.5° C./min and held for 30 min, and the final stage involves increasing the temperature to 1150° C. at a rate of 5.0° C./min. This is held for 90 min before allowing the microbeads to cool to room temperature at a rate of 10.0° C./min. By varying the sintering profile, the integrity of the microbeads can be altered.

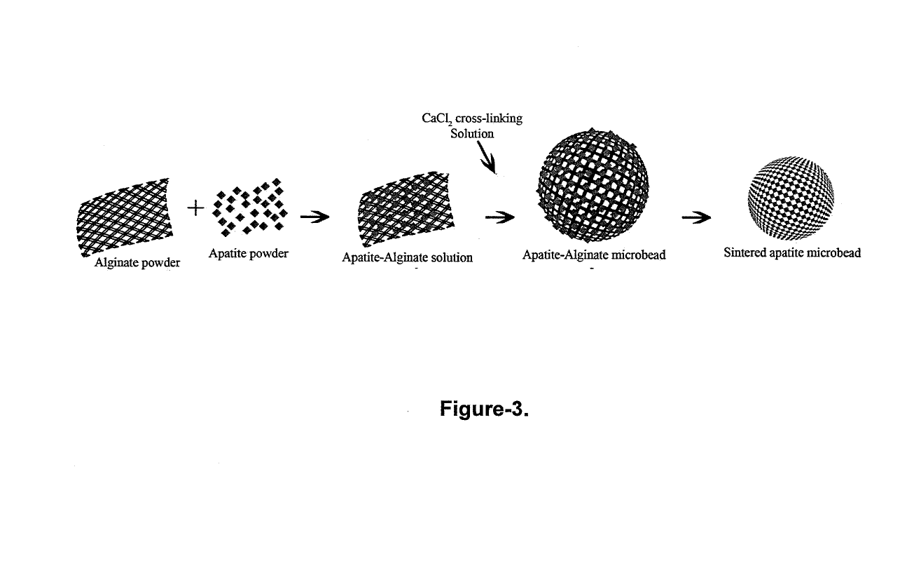

FIG. 3. Process of microbead fabrication. Alginate functions as the matrix for apatite powder. The extrusion process allows spherical microbeads to be formed whilst calcium chloride allows for ionotropic gelation between the alginate G-blocks and Ca2+ ions. The microbeads are then subjected to a sintering process whereby alginate is burnt off, leaving pure apatite microbeads.

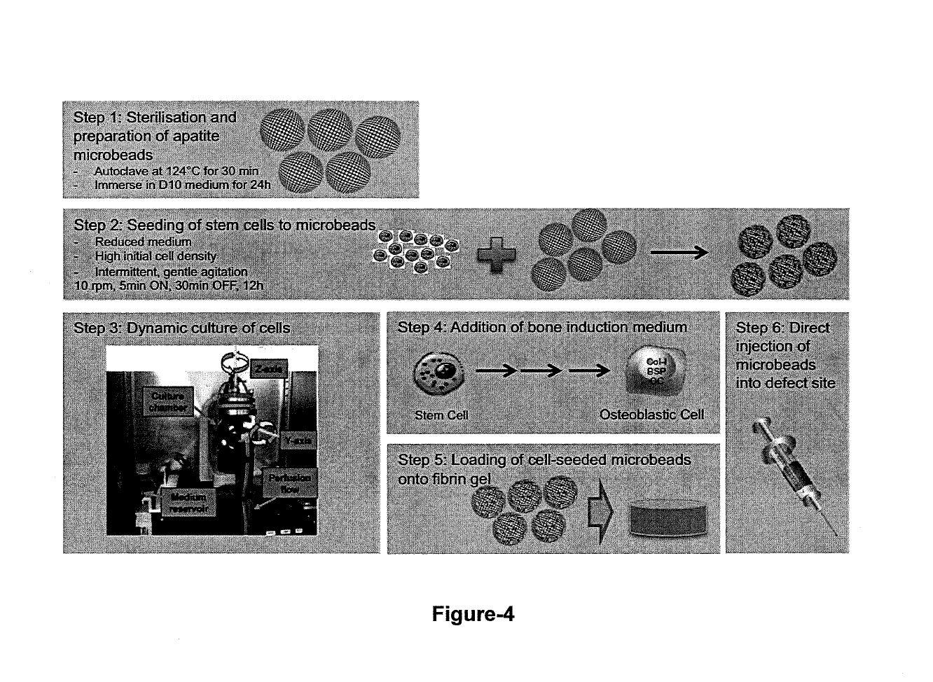

FIG. 4. Implementation of apatite microbeads as a 3D environment for cell culture.

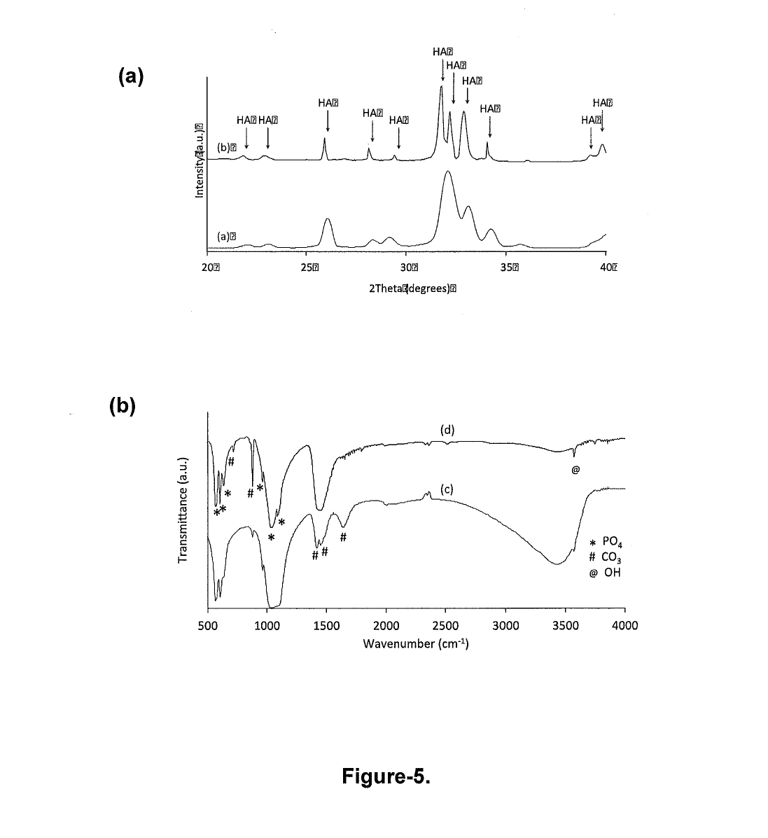

FIG. 5. XRD patterns of (a) as-synthesised apatite powder and (b) sintered apatite microbeads. Arrows correspond to apatite (JCPDS 09-0432). Sintered apatite microbeads display high purity and crystallinity.

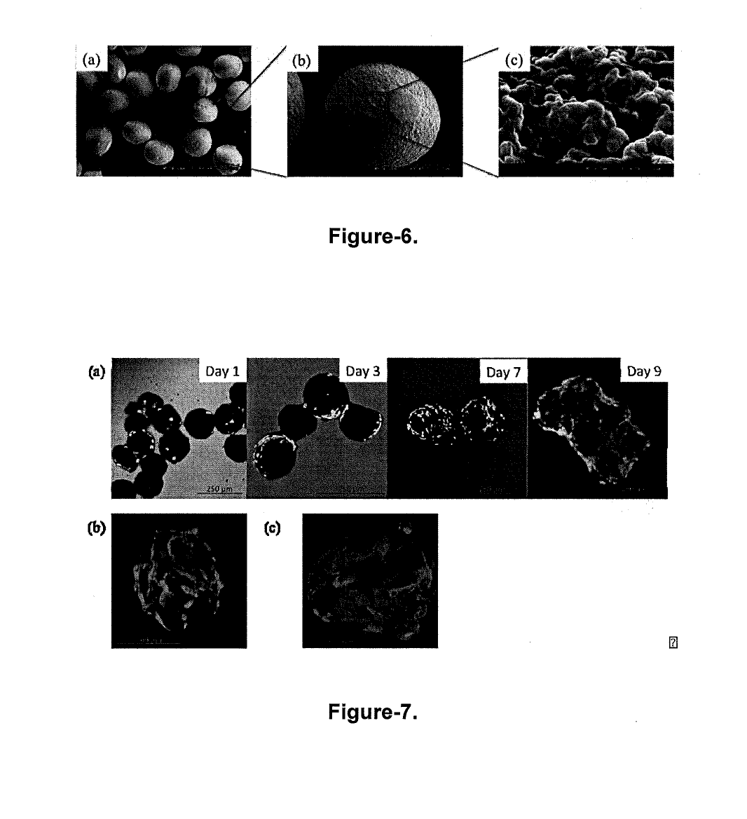

FIG. 6. SEM images of sintered apatite microbeads. (a) Apatite microbeads of diameter ˜200 μm were synthesized. (b) Apatite microbeads have a rough surface. (c) High magnification reveals that these microbeads are porous and interconnected.

FIG. 7. (a) CLSM images of MSCs seeded on apatite microbeads at day 1, 3, 7 and 9 using FDA/PI staining. Live and dead cells are stained green and red, respectively. Some microbeads are devoid of cells due to the nature of static seeding condition, causing incomplete distribution of cells across the microbeads. Between day 3 and 9, MSCs assume a fibroblastic morphology. (b,c) Phalloidin-DAPI staining is used. Actin filaments are stained red, and nuclei stained blue. (b) CLSM image showing extensive cell coverage over an entire microbead. Actin filaments are aligned along the curvature of the microbead, demonstrating good cell adhesion characteristics. (c) Image of a 3-microbead aggregates. Cells tend to form bridges across each other, creating an interconnected network between microbeads.

FIG. 8. (a) PrestoBlue Cell proliferation assay measures the metabolic activity of MSCs, which is closely related to the cell proliferation rate. Initial seeding density for both Cytodex 3 and apatite microbeads are 1.0×10⁴ cells/cm², and 5.0×10³ cells/cm² for the adherent monolayer culture. A high initial seeding density was required due to the static culture condition for the microbeads. Attachment efficiency after 24 h for Cytodex 3 and apatite microbeads is 53 and 67%, respectively. Log expansion phase was achieved between day 3 and 5 for adherent monolayer culture and Cytodex 3 microbeads, and between day 5 and 7 for apatite microbeads. Proliferation of MSCs on apatite microbeads was slower between day 3 and 5 when compared to Cytodex 3 microbeads. However, apatite microbeads achieved 1.4-fold higher cell count at day 9 (* p<0.05, *** p<0.001). (b) Alkaline phosphatase (ALP) assay was performed on the adherent monolayer culture and apatite microbeads. Both adherent monolayer culture and apatite microbeads express similar ALP levels from day 1 to 9. ALP expression peaks at day 9 for the adherent monolayer culture, while ALP level continues to increase for the apatite microbeads. At day 12, ALP expression for MSCs seeded on apatite microbeads was 2.7-fold higher than that of the adherent monolayer culture (*** p<0.001). (c) Type I collagen production was measured. MSCs seeded on the apatite microbeads produced greater amount of type I collagen throughout the culture days (* p<0.05, *** p<0.001). (d) Calcium uptake by MSCs was measured by recording the amount of Ca²⁺ ions in the initial bone-induction media, and subtracting it with the level of Ca²⁺ at each time point. Calcium uptake was higher for MSCs seeded on apatite microbeads at all the time points (* p<0.01, ** p<0.001). Calcium uptake for the control (not shown) is statistically insignificant (p>0.05). The control consists of apatite microbeads in the same condition, but without any MSCs.

Table 1. FTIR peak positions of as-synthesised apatite powder and sintered apatite microbead, referenced to peal assignments of pure HA.

### Materials and Methods

### Apatite Synthesis

In this study, a stoichiometric apatite is defined as a synthetic apatite with a Ca/P atomic ratio that approaches 1.67. Any form of apatite can be used, such as, but not limited to pure hydroxyapatite, Silicon-substituted apatite, Silver-substituted apatite, Magnesium-substituted apatite, the choice of which is dependent upon the particular application. Orthophosphoric acid (H₃PO₄) solution was added drop-wise to calcium hydroxide [Ca(OH)₂] under continuous stirring at room temperature whilst the pH was adjusted to above 10.5 by the addition of aqueous ammonia. Stirring was maintained for a further 16 h. Precipitate was further aged for 14 days before washing with distilled water, and subsequently dried.

### Preparation of Apatite Microbeads

The process of fabricating phase-pure apatite microbeads is summarised in FIG. 1. A sodium salt of alginic acid obtained from brown algae (Aldrich, 180947) was used, having a M/G ratio of 1.56 (61% mannuronic, 39% guluronic acid). Alginate was dissolved in deionised water until total homogenisation at a concentration of 0.03 g/ml.

Apatite powder was added to the alginate solution in a proportion of 50 wt % until thorough dispersion. Camphene, serving as a porogen, may be added in a proportion of 10-30 wt % to increase the desired porosity level of the microbeads. The resulting suspension was extruded drop-wise through an air-pressure assisted, electrical valve controlled droplet device (drop-on-demand) where spherical droplets were allowed to fall onto a 0.5 M calcium chloride cross-linking bath (FIG. 2a). Droplets were extruded at a rate of 200 drops/min. Upon landing into the CaCl₂ solution, spherical beads with an average diameter of 450 μm formed instantaneously due to the crosslinking reaction. The microbeads were washed twice in de-ionised water before immersing in 20 vol. % Tween 20 to lower the surface tension, thus dispersing the microbeads. This was then followed by immersion in isopropyl alcohol to harden the microbeads, followed by treatment with hexane to dry them. The dried, separated alginate-apatite microbeads were then subjected to a multi-stage sintering process to 1150° C. for 2 h in air to burn-off the alginate, thereby producing phase-pure sintered apatite microbeads. The detailed sintering process is shown in FIG. 2b.

### Characterisation of Apatite Microbeads

Surface features of the apatite microbeads were studied using field emission scanning electron microscopy (FESEM, Hitachi S-4300). Samples were gold sputtered for 15 s at 20 mA and viewed at an accelerating voltage of 15 kV. The crystallographic information of as-synthesised apatite powder and sintered apatite microbeads was investigated using powder X-ray diffraction (XRD, Shimadzu X-ray diffractometer, Model 6000). The microbeads were crushed and compacted before loading onto the machine. CuKα radiation (λ=1.5406 Å) at a scanning rate of 0.3°/min was used over a 20 range of 20-40° with a sampling interval of 0.05° at 30 mA and 40 kV. Phases were identified by comparison of the experimental data with the reference data from the International Centre for Diffraction Data (JCPDS). To ensure no alginate functional groups are present after the sintering process, fourier transform infrared spectroscopy (Varian 3100 FTIR spectrometer) was used. For this purpose, the sintered HA microbeads were crushed to powder and analysed. A wavelength range of 400 to 4000 cm⁻¹ was used, and 3 readings were taken for each sample to even out irregularities.

### MSC Isolation

Single-cell suspensions of foetal bone marrow were prepared by flushing the marrow cells out of humerus and femurs using a 22-gauge needle into Dulbecco's modified Eagle's medium (DMEM, Sigma, USA)-GlutaMAX (GIBCO, USA) supplemented with 10% fetal bovine serum (FBS), 50 U/ml penicillin, and 50 mg/ml streptomycin (GIBCO, USA) (referred as D10 medium), and then plated onto 100 mm dishes at 106 mononuclear cells/ml in D10 medium. Media was changed every 2-3 days and non-adherent cells were removed, and sub-cultured at 104/cm² to sub-confluence. MSCs at passage 3 were used in this study.

### Implementation of Microbeads and Use as Scaffold for MSC Culture

FIG. 4 presents the implementation of the sintered apatite microbeads. Apatite microbeads were firstly sterilised by autoclaving at 124° C. for 30 min before immersing in D10 medium (DMEM supplemented with 10% fetal bone serum and 1% penicillin streptomycin) for 24 h. Stem cells (e.g. human fetal mesenchymal stem cells, hfMSCs) were added at a density of 1.0×10⁵ cells/ml to 2 mg/ml of apatite microbeads. The cell-microbead suspension was held in a spinner flask, and subjected to gentle intermittent agitation cycle at 10 rpm for 5 min followed by 30 min rest, for 12 h. After the initial attachment regimen, cell-seeded microbeads were transferred to a bi-axial bioreactor, and cultured for 7 days. D10 medium was changed every 3-4 days. This was followed by changing the cell medium from D10 to bone induction medium (D10 medium supplemented with 10 mM β-glycerophosphate, 10⁻⁸ M dexamethasone and 0.2 mM ascorbic acid). By doing so, this promoted osteogenic differentiation of the stem cells which was verified by analyzing the ALP expression, type I collagen production, and calcium uptake from the cell medium. Once osteogenic differentiation was confirmed, the cell-seeded microbeads were then loaded onto fibrin gel, and introduced into the patient's defect site through the use of a 22-gauge syringe needle.

### Cytocompatibility Study

18 mg of apatite microbeads and 3.7 mg of Cytodex 3 microbeads (GE Healthcare, USA) were added to each well. The calculation of weights used was based on the total surface area added per well. For the apatite microbeads, this was 0.55 cm²/mg, and 2.7 cm²/mg for Cytodex 3. This allowed for the total surface area per well of each microbead type to be 10 cm². A total of 1.0×10⁵ cells were then added to each well, such that the seeding density for both microbead type was 1.0×10⁴ cells/cm². For the adherent monolayer culture, 1.0×10⁴ cells were added to each 24-well plate such that seeding density was 5.0×10³ cells/cm². The difference in seeding density between microbead culture and the adherent monolayer culture was to account for the low seeding efficiency of the microbeads under static conditions, such that after 24 h, cells attached on all three surfaces were of similar density. Cell viability was assessed quantitatively using the PrestoBlue assay (Invitrogen, USA) which measures cell viability through the reduction of resazurin to resorufin. On the designated time points, 10% PrestoBlue reagent was added to each well and incubated for 25 min at 37° C. Each time point was measured in triplicates. Absorbance at 570 nm, referenced at 600 nm was read using a microplate reader (Tecan, USA). The intensity cross-referenced to a standard calibration curve of MSC count against absorbance was recorded at the beginning of the study to obtain the live cell count at each time point. The qualitative analysis of cell viability on apatite microbeads was performed by fluorescein di-acetate/propidium iodide (FDA/PI) staining, where FDA stains viable cells green, and PI stains necrotic and apoptotic cell nuclei red. Apatite microbeads at different time points were retrieved from the well, rinsed with PBS and stained with FDA/PI, and viewed under a confocal laser scanning microscope (CLSM, Olympus FV1000, Japan). Cellular behaviour was assessed qualitatively by examining cytoskeletal network for any abnormalities. On day 9 of cell culture, apatite microbeads were retrieved from the well and stained with Phalloidin-DAPI, and viewed under CLSM. Actin filaments are stained red, while nuclei stained blue.

### Osteogenic Differentiation Study

On day 7 of cell culture, MSCs cultured on both apatite microbeads and the adherent monolayer were induced to differentiate down the osteogenic lineage by replacing the D10 medium with bone induction media (D10 medium supplemented with 10 mM β-glycerophosphate, 10-8 M dexamethasone and 0.2 mM ascorbic acid). Media was changed every 2-3 days. Alkaline phosphatase (ALP) plays a key role in signal transduction and cellular modulations. ALP was measured using SensoLyte pNPP Alkaline Phosphatase Assay Kit (AnaSpec USA). MSCs cultured on both apatite microbeads and the adherent monolayer were lysed using the provided lysis buffer and Triton X-100. The cell suspension was incubated at 4° C. for 10 mM under agitation and centrifuged at 2500 rpm for 10 min. The supernatant collected was then used for ALP assay. The level of ALP activity was determined by absorbance measurements at 405 nm using p-nitrophenyl phosphate (pNPP). ALP levels were normalized to the total cell count. As the primary organic constituent of bone, type I collagen level has been linked to bone growth and formation. A MicroVue CICP EIA Kit (Quidel, USA) was used to quantitatively determine the levels of C-Terminal of type I collagen (CICP) released into the media by the cells. 72 h after each media change the media of each sample was drawn and diluted 1:12 with the assay buffer.

Media was then added to coated strips of purified murine monoclonal anti-CICP antibody and incubated for 120 min at 25° C. Wells were washed twice with the wash buffer and rabbit anti-CICP was then added to each well, and incubated for another 45 min at 25° C. Wells were washed 3 times, before adding the lyophilized goat anti-rabbit IgG antibody conjugated to alkaline phosphatase and incubated for another 45 min at 25° C. After washing for 3 times, a working substrate of pNPP dissolved in a diethanolamine and magnesium chloride solution was added to each well and incubated for 30 min at 25° C. Finally, a solution of 0.5 N NaOH was added to stop the reaction and the optical density at 405 nm was read using the microplate reader. The intensity obtained from the samples was compared to a calibration curve obtained using known CICP standards to determine the concentration of CICP in the samples. CICP values were normalised to total cell count. During mineralisation, cells convert calcium ions from the surrounding media into insoluble apatite, which is deposited as ECM. The amount of calcium ions taken up from the surrounding media was measured to determine the level of mineralisation. Calcium ions in the samples were quantified using a QuantiChrom calcium assay kit (BioAssay Systems, USA). Samples were diluted and incubated with a phenolsulphonephthalein dye which formed stable blue coloured complex specifically with free calcium in the sample. After the incubation the intensity of the colour was then measured at 575 nm and calcium concentration calculated with a standard curve. Control cell-free apatite microbeads were used as negative controls. Calcium uptake was then calculated by recording the initial amount of calcium concentration from the bone induction medium, and subtracting it by the calcium concentration of the sample. Calcium uptake was normalised to total cell count.

### Statistical Analyses

Data from each time point was obtained in triplicates. All the data is represented as mean±standard deviation, and compared using either two-way ANOVA or student t-test. A value of p<0.05 was taken as significant.

### Results

### Characterisation of Apatite Microbeads

A good apatite product shall be free from impurities that will induce an inflammatory response or degrade the stability of the microbeads. X-ray diffraction (XRD) and fourier transform infrared spectroscopy (FTIR) were conducted and showed a high level of purity of a single apatite crystalline phase, without tricalcium phosphate or tetracalcium phosphate formation (FIGS. 5a and 5b). This is further confirmed via the FTIR spectra (FIG. 5) where hydroxyl, carbonate and phosphate peaks of both as-synthesised powder and sintered microbeads correspond well to the referenced apatite (Table 1). Sintering at 1150° C. resulted in the removal of water, as observed in the resolution of the broad band (3000-3800 cm⁻¹) to a sharp peak at 3572 cm⁻¹, which is characteristic of O—H bond stretching. Functional groups of alginate such as the C—H stretch (2850-3000 cm⁻¹) or C═O stretch (1722 cm⁻¹) were not present.

Morphology of the sintered apatite microbead was examined via scanning electron microscope (FIG. 6). Spherical microbeads of diameter ˜200 μm were obtained. They exhibit rough surfaces, thus favouring protein adsorption and cell attachment (FIG. 6b). At high magnification, these microbeads exhibited an interconnected porous network that extended into the core of the microbead (FIG. 6c).

Cytocompatibility of the apatite microbeads was assessed in-vitro, comparing to commercial Cytodex 3 microbeads. An initial seeding density of 1.0×10⁴ cells/cm² was used. After 24 h in culture, ˜67% of the seeded MSCs attached to the apatite microbeads as compared to ˜53% for Cytodex 3. The disparity in cell attachment is attributed to the ability of apatite material to absorb more proteins, specifically fibronectin from the serum, thus promoting greater MSC adhesion since MSCs have been reported to express fibronectin ligand receptor integrins. This is further enhanced due to the rough surface of the apatite microbeads, increasing the surface energy and thereby greater protein adsorption.

Apatite Microbeads are Bio-Compatible with MSCs and can be Used to Stimulate Osteogenic Differentiation

The viability of MSCs was assessed through FDA/PI staining. Confocal laser scanning microscope (CLSM) images showed largely viable MSCs throughout the culture days (FIG. 7a), revealing that the material composition of apatite microbeads had no cytotoxic effect. This observation was further confirmed by PrestoBlue Cell proliferation assay (FIG. 8a), showing an increase in live cells from day 1 to 9. In terms of growth kinetics, log phase growth was achieved between day 5 and 7 for apatite microbeads. On day 9, MSCs grown on apatite microbeads reached 3.0×10⁴ cells/cm² which equates to a cell density of 3.3×10⁵ cells/ml. By comparing to the adherent monolayer culture, which yielded 1.5×10⁵ cells/ml, this translates to a 2.2-fold increase in cells per unit volume. Thus, we believe that using apatite microbeads for stem cell expansion is not to increase the rate of proliferation, but rather to increase the efficiency of cell culturing by optimising cell yield per unit volume of culture medium while reducing several culture cycles.

Morphology of MSCs examined on day 1 and 3 were spheroidal in shape. From day 7 onwards, they exhibited a spindle-shaped morphology similar to those observed on adherent monolayer culture (FIGS. 7a and 7b). No abnormal cells in the form of ruptured membrane were observed. Cytoskeletal actin filaments were aligned along the curvature of the apatite microbeads (FIG. 7b), indicating good attachment and cell adhesion to the microbeads. Where microbeads overlapped, cell bridges were formed, creating an interconnected network between cells from one microbead to cells from another (FIG. 7c). Such a network is essential for maintaining proper cellular signalling, allowing for upregulation of various biomolecular chemicals thus maintaining the osteogenic potency of MSCs.

### MSC Osteogenic Potential Cultured on Microbeads is Increased Compared to Monolayer Culture

The osteogenic potential of MSCs seeded on apatite microbeads was investigated. Alkaline phosphatase (ALP) activity, type I collagen production and calcium uptake were measured at various time points, and compared to the adherent monolayer culture. Results (FIG. 8b-d) demonstrate that the osteogenic potential of MSCs seeded on apatite microbeads was enhanced as compared to that of the adherent monolayer culture. On day 12, ALP expressed by MSCs seeded on the apatite microbeads continued to increase, while those seeded on the adherent monolayer culture declined sharply after day 9 (FIG. 8b). This phenomenon suggests that MSCs cultured on apatite microbeads are able to maintain high level of ALP expression, thus promoting the mineralisation of ECM. This explanation is corroborated with type I collagen production and calcium uptake. Our results show that type I collagen production on apatite microbeads was higher throughout culture, and production level continued to rise after day 9 (FIG. 8c). Calcium uptake by MSCs seeded on apatite microbeads exhibited a similar trend, increasing till day 9 before levelling off, while the adherent monolayer culture remained low after an initial increase till day 3. At day 12, MSCs seeded on apatite microbeads exhibited ˜1.8-fold and ˜1.5-fold increase in type I collagen production and calcium uptake, respectively when compared to the adherent monolayer culture.

### Apatite Microbeads Show Superior Biological Viability Compared to Other Scaffolds

To assess the biological viability of human fetal MSCs seeded on sintered apatite microbeads quantitatively, a cell proliferation assay was conducted, and compared with Cytodex 3 and adherent monolayer culture flask. Results (FIG. 8a) obtained show higher cell attachment efficiency on apatite microbeads (67%) as compared to Cytodex 3 (50%). Exponential growth phase was achieved from day 5-7. On day 9, cells proliferating on sintered apatite microbeads was 1.4-fold higher than that of Cytodex 3 (* p<0.05, *** p<0.001). ALP assay was performed on the adherent monolayer culture flask and apatite microbeads. Both adherent monolayer culture flask and apatite microbeads (FIG. 8b) expressed similar ALP levels from day 1 to 9. ALP expression peaked at day 9 for the adherent monolayer culture, while ALP level continues to increase for the apatite microbeads. At day 12, ALP expression for hfMSCs seeded on apatite microbeads was 2.7-fold higher than that of the adherent monolayer culture flask (*** p<0.001). Type I collagen production was measured (FIG. 8c). hfMSCs seeded on the apatite microbeads produced a greater amount of type I collagen throughout culture (* p<0.05, *** p<0.001). Calcium uptake by hfMSCs was measured by recording the amount of Ca²⁺ ions in the initial bone-induction medium, and subtracting it with the level of Ca²⁺ at each time point. Calcium uptake was found to higher for hfMSCs seeded on apatite microbeads throughout culture (FIG. 8d) (* p<0.01, ** p<0.001). Calcium uptake for the control (not shown) was statistically insignificant (p>0.05).

### SUMMARY

Our results show that MSCs exhibit better osteogenic potency when cultured on apatite microbeads that mimic the chemical and morphological structure of physiological bone. Compared to conventional polymeric microbeads (Cytodex 3), our apatite microbeads are easily customizable and advantageously injectable/implantable. Their rough surface promotes cell adhesion, and the presence of regular (and customizable) interconnected pores promotes cell interaction and growth, with apatite beads exhibiting improved cell proliferation and viability. When utilized to culture bone cell, enhanced osteogenic differentiation was observed.

In conclusion, we have demonstrated a scalable method to obtain MSCs with enhanced osteogenic potency on apatite microbeads, which can be used in direct implant science. The method is simple and efficient and does not require repeated trypsinisation and replating onto multiple flasks to expand cells. This microcarrier technology certainly has the potential to accelerate bone or periodontal tissue engineering research for clinical applications and at the same time, serves as a proof-of-concept for future large-scale stem cell expansion.

TABLE 1
FTIR peak positions of as-synthesised apatite powder and sintered
apatite microbead, referenced to peal assignments of pure HA.

| | | As-synthesised | Sintered |
|---|---|---|---|
| Peak Assignment | HA (reference) | powder | Microbeads |
| Hydroxyl Stretch | 3571 | 3000-3800 | 3572 |
| Carbonate v₃ | 1650 | 1454 | 1454 |
| | 1417 | 1423 | — |
| Phosphate v₃ | 1091 | — | 1093 |
| | 1041 | 1047 | 1047 |
| Phosphate v₁ | 961 | 960 | 960 |
| Carbonate v₂ | 873 | 876 | 876 |
| Phosphate v₄ | 629 | 629 | 633 |
| | 603 | 604 | 604 |
| | 567 | 567 | 567 |

1. A microcarrier bead made from apatite and characterised by one or more, including any combination, of the following features:
a) micrometre-sized;
b) a regular porous structure;
c) rough surface;
d) substantially spherical;
e) osteo-conductivity;
f) chemical similarity to the mineral phase of natural bone; and
g) high thermal stability permitting them to be easily sterilized.

2. A microcarrier bead according to claim 1 wherein said apatite is selected form the group comprising: hydroxyapatite, silicon-substituted apatite, silver-substituted apatite, magnesium-substituted apatite and a stoichiometric apatite which is a synthetic apatite with a Ca/P atomic ratio that approaches 1.67.

3. A microcarrier bead according to claim 1 wherein said apatite is phase-pure.

4. A microcarrier bead according to claim 1 wherein said beads are between 100-800 μm diameter, or 200-600 μm diameter, or 400-500 μm diameter.

5. A microcarrier bead according to claim 1 wherein said beads have a regular pore size as observed by Scanning Electron Microscopy.

6. A microcarrier bead according to claim 1 wherein said beads can withstand temperatures up to 1500° C. for up to 10 hours.

7. A microcarrier bead according to claim 1 wherein said beads have osteogenic potency.

8. A plurality of microcarrier beads according to any one of claim 1.

9. A method for making microcarrier beads comprising:
a) mixing apatite and alginate in a solution and allowing them to disperse to form a suspension;
b) extruding said suspension drop-wise through a droplet device;
c) exposing said extruded droplets to calcium chloride (CaCl₂) solution;
d) washing said beads to remove said CaCl₂ solution and dispersing same;
e) hardening the beads in a solution of alcohol;
f) drying the beads; and
g) sintering the beads to burn of the alginate.

10. The method according to claim 9 wherein in part b) the solution contains a porogen.

11. The method according to claim 9 wherein under part g) the beads were subjected to a multi-stage sintering process which took the temperature to 1150° C. for 2 h in air to burn-off the alginate.

12. A therapeutic comprising a microcarrier bead according to claim 1 and attached thereto or grown thereon at least one selected cell or tissue type.

13. A therapeutic according to claim 12 wherein said cell or tissue is selected from the group comprising: a stem cell, progenitor cell and induced pluripotent stem cell.

14. A therapeutic according to claim 13 wherein said stem cell is a human cell.

15. Use of a microcarrier bead according to claim 1 for the repair of craniomaxillofacial defects, wrist fractures, spinal fusion procedures ad periodontal defects.

16. A method for making a therapeutic according to claim 12 comprising;
a) mixing microcarrier beads according to claim 1 with at least one selected cell or tissue type in solution to form a suspension;
b) agitating said suspension to encourage said cells or tissue to attach to said beads;
c) culturing said cell attached beads to encourage growth of said cells or tissue; and
d) adding said cell-attached beads to a carrier gel.

17. A method according to claim 16 wherein said beads are sterilized prior to use.

18. A method according to claim 16 wherein said cell or tissue is selected from the group comprising: a stem cell, progenitor cell and induced pluripotent stem cell.

19. A method according to claim 18 wherein said stem cells were added at a density of 1.0×10⁵ cells/ml to 2 mg/ml of apatite microbeads.

20. A method according to claim 16 wherein step c) above involves exposing said cells or tissue to induction or differentiation medium.

21. A method according to claim 20 wherein said medium is bone induction medium (D10 medium supplemented with 10 mM β-glycerophosphate, 10⁻⁸M dexamethasone and 0.2 mM ascorbic acid).

22. A method of treatment involving the use or administration of the microcarrier beads according to claim 1.

23. A method according to claim 22 wherein said beads are used to treat a condition selected form the list comprising: cranio-maxillofacial defects, wrist fractures, spinal fusion procedures and periodontal defects.
